(19) United States
(12) Patent Application Publication
(10) Pub. No.: US 2004/0013740 A1
(43) Pub. Date: Jan. 22, 2004

(54) Dendritic cell loaded with toxic substances

(76) Inventor: Dirk Weickmann, Munchen (DE)

Correspondence Address: JENKINS & WILSON, PA, 3100 TOWER BLVD, SUITE 1400, DURHAM, NC, 27707, US

(21) Appl. No.: 10/292,282

(22) Filed: Nov. 12, 2002

Related U.S. Application Data
(63) Continuation of application No. PCT/EP01/05670, filed on May 17, 2001.

(30) Foreign Application Priority Data
May 17, 2000 (DE) 10024383.5

(51) Int. Cl. A61K 35/30; C12N 5/00

(52) U.S. Cl. 424/570; 435/373

(57) ABSTRACT

The invention describes dendritic cells loaded with toxic substances wherein the toxic substances are selected from the poison of scolopenders of the genera Scolopendra and Hemiscolopendra, snakes of the genera Bitis and Naja, spiders of the genera Loxosceles, Sicarius and Pholcus and Dysdera as well as scorpions of the genus Parabuthus and a combination of one or more of these toxins.

### SUMMARY

The invention relates to dendritic cells loaded with toxic substances, methods for the preparation thereof as well as to the use of said dendritic cells for the treatment of tumors, particularly for the therapy of tumors of the mucosa and the skin.

### PRIOR ART

Skin cancer refers to tumors occurring in the skin and less frequently in the mucosa. The malignant tumors are designated as cutaneous carcinomas or melanomas, respectively. The primary tumors are manifested in the skin and can be recognized at the surface of the skin as pigmented areas (Veronesi U., Cascinelli N., Santinami M. (1987): Cutaneous Melanoma. Status of Knowledge and Future Perspective. London Orlando San Diego New York Austin Boston Sidney Tokyo Toronto: Academic Press). To distinguish them from naevi with which they are frequently mixed up due to their appearance tissue sampling and in many cases also growth in tissue culture is required. The most aggressive among the known neoplasias is malignant melanoma, often also called “black cancer”. A problem with this disease relates to its extreme metastatic spreading which is stopped by any organ boundary.

At present the following therapies are preferred:

Currently, excision of the cutaneous tumor is the method most widely used. Due to the depth or the spreading, respectively, sufficient dissection of the cutaneous tumor is impossible.

Therefore a combination with other types of therapy is often attempted (Voigt H. (1996): 150 Fragen und Antworten zum malignen Melanom. Zuckschwerdt Verlag, München).

Disadvantages:

In-hospital treatment and/or anesthesia, wound healing and scar formation.

Radiotherapy:

At present radiotherapy is of decreasing importance in the case of skin cancer and is only used for the treatment of lymph node and cutaneous tumor metastases.

The irradiation is carried out by means of ionizing radiation wherein generally electron, gamma, neutron, or X-ray beams are used (Zetkin/Schaldach: Lexikon der Medizin, 16^{th} edition, 1999, page 1922/1923, Ullstein Medical).

Similar to chemotherapy, irradiation has the disadvantage that it is impossible to achieve a spatial restriction. Because of the intensity of the radiation also healthy cells and particularly the DNA are severely damaged. Since cancer cells generally divide faster than normal cells, under typical circumstances the cancer cells are the first to be destroyed in radiotherapy. However, there is the risk developing a radiation ulcer (Pschyrembel—Klinisches Worterbuch 256, edition 1990, page 1602).

Chemotherapy:

With respect to chemotherapy it has been found that mainly metastasing melanomas almost do not respond to chemotherapy. A monotherapy with cytostatics is generally insufficient so that polychemotherapies are primarily employed. For the most part, the following substances are used:

dacarbacine (DTIC)

cisplatin (CDDP)

carboplatin (CBDCA)

ifosfamide (IFO)

fotemustine

bendamustine

CCNU

BCNU

methyl-CCNU

cyclophosphamide (CPA)

vindesin (DVA or VDS)

vinblastin (VBL)

vincristin (VCR)

procarbacine (PBZ)

mitomycin C (MMC)

The use of these substances is performed in the following therapy regimes which currently have found to be most effective:

bleomycin+vincristin+CCNU+dacarbacine (BOLD protocol)

BCNU+cisplatin+dacarbacine+tamoxifen (+interleukin−2/interferon−alpha)

cisplatin+vinblastin+bleomycin (CVB protocol)

cisplatin+vindesin+dacarbacine (CVD protocol)

BCNU+hydroxyurea+dacarbacine (BHD protocol)

dacarbacine+vincristin+BCNU (DVB protocol)

cisplatin+ifosfamide

fotemustine+interferon−alpha

—Voigt H. (1996): 150 Fragen und Antworten zum malignen Melanom. Zuckschwerdt Verlag, München—

Besides the positive effects against the genetically defective cells, however, the chemotherapies mentioned also show many side effects and disadvantages.

Disadvantages of chemotherapy are that it is difficult to use them in a site-specific manner due to their chemical structure; these cytostatics are extraordinarily severe cell toxins which to a great extent also damage healthy tissues including liver and kidney cells in addition to the tumor tissue. The side effects arising such as for example alopecia, vertigo, vomiting, gastro-intestinal bleeding, disturbed circulation etc. are difficult to assess due to the systemic distribution of the cytostatics (Deutsches Krebsforschungszentrum DKFZ Heidelberg—Focus 19/1995).

These numerous, dangerous and undesirable side effects may be explained mainly by an inhibition of the regeneration of quickly proliferating tissues and they particularly affect the hematopoietic system, the mucosal and gonadal epithelia as well as the skin and skin appendages. Among the life-threatening complications infections are the most important, followed by bleeding (Pschyrembel—Klinisches Worterbuch, 256^{th} edition, 1990, page 1866).

Cytokin Treatment:

In this respect it has been mainly attempted to employ the interferon therapies which up to now are only approved for indications of Kaposi sarcoma and hairy cell leukemias. Interferon belongs to the cytokins which are actively effective against malignant cells. The mode of action is based on the recognition of surface proteins of cancer cells and on their destruction.

Among the disadvantages of interferon therapy are fever, anorexia-nausea-emesis syndrome, fatigue, head stress, headache, joint ache, vertigo, increase in liver enzymes, pruritis, IFN antibody formation, circulation and blood pressure problems (DeVita jr. V T., Hellmann S., Rosenberg S A. (1991): Biologic Therapy of Cancer. Philadelphia: J. B. Lippincott Co.).

Antibody Therapy:

Besides interferon therapy, monoclonal antibodies are preferably used for the treatment in immunotherapies.

For this purpose, myeloma cells grown according to a method by Köhler and Milstein are fused to rodent lymphocytes immunized with human antigenic melanoma cell material or membrane fractions of melanoma cells. This results in a nearly infinite amount of monoclonal antibodies from melanoma cells. Then, the antibodies are injected and find and destroy the cutaneous carcinoma cells in a natural way by means of immune modulators. No side effects are known because they belong to the body's own substances so that no immune shock reactions occur. The disadvantage of this type of therapy is, however, the low sensitivity of the cutaneous carcinoma cells (Voigt H. (1996): 150 Fragen und Antworten zum malignen Melanom. Zuckschwerdt Verlag, München/Ibelgaufts H. (1992): Lexikon Zytokine. Medikon Munich).

Therapy Using Dendritic Cells:

Since about 1995, much research effort has been spent regarding the possibility of using the body's own dendritic cells in the treatment of skin cancer. For this purpose, dendritic cells are grown in vitro from spinal cord stem cells and/or blood stem cells of the patient which are then after a certain minimal titer has been achieved are injected back into the patient. The dendritic cells actively seek and find tumor cells which they attack with cell destructive substances. In this respect, the low efficacy is the main disadvantage and it is attempted to load the dendritic cell with the body's own human cytokins and/or peptides (e.g. anaphylatoxins C3a, C4a etc.) (Kirchner H., Kruse A., Neustock P., Rink L. (1994): Cytokine und Interferone. Spektrum Akademischer Verlag, Heidelberg). Unfortunately, the substances found often have an insufficient specificity to completely destroy the tumor cells.

From the publication of the Journal of Investigative Dermatology (January 2000), volume 114, No. 1, page 234, abstract No. 47, the use of cholera toxin as an adjuvant in experimental animals is known. The toxic subunit of cholera toxin (CTA) was separated from the CTB subunit which is responsible for the binding of cholera toxin to the cell membrane and the CTB subunit is used to induce a humoral immune response. The loading according to the present invention of dendritic cells with toxic substances, particularly with those mentioned in claim 1, is not anticipated or rendered obvious by this. The toxins are not used as adjuvants by the invention but to direct the toxic substances by means of the dendritic cells to the tumor cells in a targeted manner.

Therefore, it is an object of the present invention to provide improved means for the therapy of tumor diseases which can be attacked by means of dendritic cells.

According to the present invention this object has been achieved by loading dendritic cells with toxic substances wherein the toxic substances are selected from the poison of scolopenders of the genus Scolopendra and Hemiscolopendra, snakes of the genus Bitis and Naja, spiders of the genus Loxosceles, Sicarius and Pholcus and Dysdera as well as scorpions of the genus Parabuthus and from a combination of one or more of these toxins.

Dendritic cells loaded in this way are useful in the therapy of tumor diseases, particularly in the therapy of cutaneous cancer and mucosal tumors. The dendritic cells may be used as the only therapy but also as a supportive therapy without risking the disadvantages of the current state of the art.

In the following the invention will be described in more detail. However, the invention is not limited to the following specification and to the Examples. The persons skilled in the art are able to modify the invention on the basis of the accompanying claims considering the description without leaving the scope of the accompanying claims.

Loading of the dendritic cells is performed with one or more toxic substances from the poison of scolopenders of the genus Scolopendra and/or Hemiscolopendra, snakes of the genus Bitis and Naja, as well as scorpions of the genus Parabuthus and/or spiders of the genus Loxosceles, Sicarius and Pholcus in a pharmaceutically and therapeutically effective amount. It is also possible to use at least two of these substances from the poisons.

The poison of the animals mentioned above contains a whole cocktail of compounds comprising not only peptide toxins but also toxins with an amido group which however have no protein structure and have for example a molecular weight of 30-35 kDa. Furthermore the poison cocktail also includes substances having an antagonistic effect which ensure a spatially and temporally controlled distribution of the poisons released by the animal.

The toxins preferably are peptide toxins. The toxins generally have necrotic, cytotoxic and/or apoptotic properties. The toxins may be isolated from the poison cocktail of the animals by methods known per se. Because this requires a lot of effort, it has been found according to the present invention that after a fractionation of the poison cocktail also individual fractions containing the toxins may be used for loading of the dendritic cells. These fractions contain the toxins together with other substances. However, since the fractions are selected to contain in particular the substances having a cytotoxic, apoptotic and/or necrotic effect purification of the toxins up to complete purity is not absolutely required. If this is the case, however, and in addition also the structure of the toxins is known these may be used not only in an isolated, pure form but also in recombinant form in the case of peptide toxins. Generally, the peptide toxins have a molecular weight in the range of 50-350 kDa, preferably a molecular weight of about 100 kDa. The toxins may also be prepared synthetically, and it should be understood that also modifications of the toxins present in the animal species mentioned and used for loading of dendritic cells according to the invention are possible, i.e. derivatives obtained by chemical modification of the toxins. In the case of the peptide toxins such derivatives are meant to include also such toxins in which one or more amino acids have been added, deleted and/or substituted by other amino acids wherein it should be understood that in each case the toxic properties are retained. It should be noted that one toxin can have not only cytotoxic but also necrotic and/or apoptotic properties at the same time.

The dendritic cells loaded with toxic substances may be used in the targeted therapy of tumors. In this respect the toxins have cytotoxic effects. Since the dendritic cells actively seek and find tumor cells and attack them with cell destructive substances, the toxic substances are selected to have cell destructive, i.e. cytotoxic, necrotic and/or apoptotic properties. These are specifically the substances present in the poison cocktail of the animal genera mentioned in claim 1. Therefore, they are excellently useful for the therapy of tumors, particularly for the therapy of tumors of the skin and the mucous membranes. It is also possible to load the dendritic cells loaded in this way with further substances useful in tumor therapy, for example with cytokins, cytokin receptors, anaphylatoxins, interferons, antibodies, glycosides etc.

Loading of the dendritic cells is carried out by direct injection of the toxic substances into the cell, by incubation of the dendritic cells with the toxic substances or by lipofection. Originally, this technique has been used for the transformation of cells with foreign DNA or RNA. An enhanced efficiency is achieved by linking antibodies, proteins, glycolipids etc. to the liposomes thus influencing the interactions between liposomes and cell surfaces in a targeted manner. In this way the peptide toxins or cytokins can be recognized as “belonging to the cell” and can be introduced into the cell during “normal” metabolic functions. [Herder (1995): Lexikon der Biochemie und Molekularbiologie, volume 2, Spektrum Akademischer Verlag, Heidelberg].

Another method of loading is laser microinjection. In this method dendritic cells present in a medium containing the toxic substances are treated with a highly focused laser beam for a short time generating a hole in the cell membrane through which the surrounding medium may enter into the cell. The technique underlying this method of micromanipulation of cells or cell aggregates is described for example in Schütze K. et al.; Nature Biotechnology, 16, 8: 737-742 (1998), Schütze K. et al.; J. Cell. Mol. Biol., 44, 5: 735-746 (1998), Böhm M. et al.; Americ. J. Pathology; 151, 1: 63-67, Pontén F. et al.: Mutation Research Genomics, 382: 45-55 (1997), Bernsen M. et al.; Lab. Invest. 78: 1267-1273 (1998) or Fink L. et al.; Nat. Medicine. 4, 11: 1329-1333 (1998).

It is preferred that the peptide toxin used to load the dendritic cell and/or the substances optionally contained therein are derived from the poison of the scolopender centipede species Scolopendra gigantea ssp., Hemiscolopendra spp., of the snake species Bitis arietans (puff adder), Bitis gabonica (Gaboon viper), Bitis nasicornis (rhinoceros viper), and of the smaller Bitis species B. atropos, B. caudalis, B. peringueyi, and of the spitting cobra species Naja melanoleuca, Naja pallida and Naja naja sputatrix, the Parabuthus scorpion species Parabuthus transvallicus, Parabuthus granulosus, Parabuthus villosus, the Loxosceles spider species Loxosceles laeta, Loxosceles spiniceps, Loxosceles bergeri, Loxosceles parami, Loxosceles rufescens, Loxosceles reclusa, Loxosceles deserta, the six eyed sand/crab spider species Sicarius hahni, Sicarius albospinosus, Sicarius oweni, Sicarius testaceus, Sicarius argentinensis and/or the vibrating spider species Pholcus phalangioides, Pholcus opilionides, Pholcus spp. (RSA) and Pholcus spp. (Cuba), wherein it is particularly preferred that the peptide toxin and/or the optionally present further substances are derived from the poison of scolopenders, Bitis arietans and/or Loxosceles spiniceps. This has the advantage that the naturally present skin and/or tissue destructive effect of the substance may be utilized. In this respect it is preferred that the animal poison raw mixture is obtained from male and/or female scolopenders of the genera Scolopendra and Hemiscolopendra of a body length of more than about 15 cm, male and/or female snakes of the genera Bitis and Naja, preferably of a body length of more than 50 cm, female subadult and/or adult scorpions of the genus Parabuthus, and/or from female subadult or adult spiders of the genus Loxosceles and/or Pholcus and/or Sicarius. This is advantageous because female Arachnidae of the genera Parabuthus, Loxosceles, Pholcus and Sicarius produce a higher amount of poison than the males. In the case of scolopenders and snakes both sexes produce the same poison quantities and qualities. This has the advantage of a particularly careful preparation of the animal poison raw mixture.

Moreover, it is preferred in the method according to the present invention to homogenize the animal poison raw mixture prior to fractionation, and it is further preferred that the fractions are deep-frozen and further preferred lyophilized prior to processing. The pharmaceutical loading of the dendritic cells according to the present invention is suitable for use in medicine. According to the invention the dendritic cells loaded with pharmaceutically effective substances may be used preferably in the treatment of cutaneous cancer diseases. Further preferred is the use of a peptide toxin from the poison of the scolopender centipede species Scolopendra gigantea ssp., Hemiscolopendra spp., of the snake species Bitis arietans (puff adder), Bitis gabonica (Gaboon viper), Bitis nasicornis (rhinoceros viper), and of the smaller Bitis species B. atropos, B. caudalis, B. peringueyi, and of the spitting cobra species Naja melanoleuca, Naja pallida and Naja naja sputatrix, the Parabuthus scorpion species Parabuthus transvallicus, Parabuthus granulosus, Parabuthus villosus, the Loxosceles spider species Loxosceles laeta, Loxosceles spiniceps, Loxosceles bergeri, Loxosceles parami, Loxosceles rufescens, Loxosceles reclusa, Loxosceles deserta, the six eyed sand/crab spider species Sicarius hahni, Sicarius albospinosus, Sicarius oweni, Sicarius testaceus, Sicarius argentinensis and/or the vibrating spider species and the Pholcus spider species Pholcus phalangioides, Pholcus opilionides, Pholcus spp. (RSA) and Pholcus spp. (Cuba) in a pharmaceutically effective loading of dendritic cells for the treatment of cutaneous carcinoma diseases, wherein peptide toxins from the poison of scolopenders, Bitis arietans and/or Loxosceles spiniceps are particularly preferred. Furthermore the use of a peptide toxin from the poison of the scolopender centipede species Scolopendra gigantea ssp., Hemiscolopendra spp., of the snake species Bitis arietans (puff adder), Bitis gabonica (Gaboon viper), Bitis nasicornis (rhinoceros viper), and of the smaller Bitis species B. atropos, B. caudalis, B. peringueyi, and of the spitting cobra species Naja melanoleuca, Naja pallida and Naja naja sputatrix, the Parabuthus scorpion species Parabuthus transvallicus, Parabuthus granulosus, Parabuthus villosus, the Loxosceles spider species Loxosceles laeta, Loxosceles spiniceps, Loxosceles bergeri, Loxosceles parami, Loxosceles rufescens, Loxosceles reclusa, Loxosceles deserta, and the Pholcus spider species Pholcus phalangioides, Pholcus opilionides, Pholcus spp. (RSA), Sicarius hahni, Sicarius albospinosus, Sicarius testaceus, Sicarius oweni, and Sicarius spp. (South America) in medicine is considered according to the present invention.

Preferably, the peptide toxin is obtained by a fractionation procedure and it is further preferred that the substance having a pharmaceutical effect is obtained from the poison cocktail of one of the species mentioned above. This ensures that the pharmaceutical efficiency of loading may be adapted in its effect in an advantageous manner to the tumor type and/or size to be treated.

The peptide toxin may be obtained from the animal poison raw mixture (whole poison cocktail) by fractionation procedures known per se for the separation of proteins. It is preferred to obtain the peptide toxin by gel chromatography, HPLC, affinity chromatography and/or ion exchange chromatography.

It is also possible, however, to prepare the peptide toxin and/or the other substances used from the poison of scolopender centipede species Scolopendra gigantea ssp., Hemiscolopendra spp., of the snake species Bitis arietans (puff adder), Bitis gabonica (Gaboon viper), Bitis nasicornis (rhinoceros viper), and of the smaller Bitis species B. atropos, B. caudalis, B. peringueyi, and of the spitting cobra species Naja melanoleuca, Naja pallida and Naja naja sputatrix, the Parabuthus scorpion species Parabuthus transvallicus, Parabuthus granulosus, Parabuthus villosus, the Loxosceles spider species Loxosceles laeta, Loxosceles spiniceps, Loxosceles bergeri, Loxosceles parami, Loxosceles rufescens, Loxosceles reclusa, Loxosceles deserta, the six eyed sand/crab spider species Sicarius hahni, Sicarius albospinosus, Sicarius oweni, Sicarius testaceus, Sicarius argentinensis, and/or the Pholcus spider species Pholcus phalangioides, Pholcus opilionides, Pholcus spp. (RSA) and Pholcus spp. (Cuba) by chemical synthesis or by suitable methods of genetic engineering in a recombinant form. As it is typical for chemical substances, the present invention comprises also derivatives and salts of the substances provided by the invention. For example the peptide toxin may comprise one or more additions, substitutions and/or deletion of amino acids while it should be ensured that the medical-pharmaceutical effect according to the present invention is preserved.

It is further preferred that for the pharmaceutically effective dose for loading the peptide toxin is present in an amount that only a controlled delivery of the peptide toxin by he dendritic cell occurs.

Further preferred is a pharmaceutically effective loading wherein the amount of peptide toxin which can be delivered by the dendritic cell is subject to a spatially and temporally controlled distribution with respect to its cell destructive effect. The pharmaceutical composition of the freight preferably comprises an amount of peptide toxin which is selected dependent on the cutaneous carcinoma and/or other tumor to be treated.

Growth of the Dendritic Cells:

Preferably the human spinal cord stem cells are deep-frozen in Eppendorf sample vials (E cups) having a capacity of 1 ml. Preferably this is a saturated stem cell solution (saline solution or plasma, 50 microliters at maximum). For further processing, preferably 750 microliters of medium (37 degrees Centigrade) consisting of 500 ml DMEM Ham's F-12, 50 ml RPMI 1260, 10 ml penicillin/streptomycin, 5 ml glutamine, and 50 ml FBS are added.

The thus diluted suspension is then agitated for 10-15 seconds on a vortex, lowest setting, without formation of foam to obtain homogenous mixing.

Directly afterwards the suspension is introduced into a 75 cm² cell culture flask and topped with 20 ml of the same medium (37 degrees Centigrade).

Then, two to three times careful manual swinging is carried out to achieve an optimal distribution of the spinal cord stem cells present in the suspension in the cell culture flask.

This cell culture flask is then placed in an incubator set to 37 degrees Centigrade.

It is preferable to leave the cell culture prepared for five days at a uniform temperature (37 degrees Centigrade) in the incubator.

This enables adhesion of the spinal cord stem cells to the bottom of the flask. The first visual control by an inverted microscope as to whether the spinal cord stem cells have adhered to the bottom of the flask is preferably carried out five days after placing the cell culture flask in the incubator.

It is preferable to carry out the first medium exchange under sterile conditions five days after preparation of the cell culture as follows:

The medium flask is held in a way that as much as possible of the medium contained therein is removed without simultaneously pouring out the already adhered cells. Optional residual medium is removed by gentle tapping of the bottleneck onto a pad.

The medium collected in this manner is discarded.

Subsequently, 20 ml of new medium are carefully introduced into the cell culture flask without releasing the adhered cells from the bottom of the cell culture flask.

The thus described medium exchange is to be carried out after every 3 or 4 days, respectively, until several cell layers have grown on top of each other and the first cells start to detach into the supernatant medium.

Growth of the cell culture is monitored by an inverted microscope prior to each medium exchange.

If the flask is overgrown with several cell layers at the bottom of the cell culture flask all the supernatant of about 20 ml is subcultured in equal amounts in 4 small 25 cm² flask and afterwards topped with medium prewarmed to 37 degrees Centigrade.

These subcultures are left for five days at 37 degrees Centigrade in an incubator without any further treatment.

Starting on the fifth day the medium exchange described above is carried out every three or four days, respectively, in each case with 5 ml medium under permanent microscopic monitoring.

If the bottom of the cell culture flask is completely overgrown with cell layers, addition of the growth factors is performed in an amount that a concentration of 900 U/ml each of GM-CSF and IL-4 as well as of 700 U/ml sIL-4R (obtained e.g. from Biochrom company) is achieved. A temperature of 37 degrees Centigrade is preferred during addition. The addition of the growth factor mixture is done with a sterile 2 ml pipette.

The cell culture flask is carefully swung manually without detaching the cells from the bottom. For at least 24 hours the cell culture flask is stored at 37 degrees Centigrade in the incubator.

Not before the elapse of 24 hours a first visual control is performed using an inverted microscope whether dendritic cells have already differentiated which are then transferred daily under highly sterile conditions using a glass Pasteur pipette with an as low amount of medium as possible into a new flask (25 cm²) containing medium (2 ml) already prewarmed to 37 degrees Centigrade until no new dendritic cells develop within the original flask.

The medium exchange for the dendritic cells collected in the new cell culture flask is preferably carried out in a manner that the old medium is aspirated with a Pasteur pipette under highly sterile conditions until only 1 ml is left without aspirating dendritic cells at the same time and is then topped with 4 ml of new medium prewarmed to 37 degrees Centigrade.

This medium exchange is subsequently performed every three or four days, respectively, in the manner described.

By the dendritic cells thus obtained having a viability of at least three weeks a sufficient number for therapeutic purposes is provided.

The dendritic cells recognize genetically defective cells of the own body, dock to these cells and deliver the active freight substance into the transformed cell. The freight achieves a targeted destruction of the malignant cell by means of its cytotoxic effect. Furthermore, dendritic cells are capable of mediating information about the tumor situation (by recognition of cytokins) but also information about tumor destructive substances to T cells. The T cells “informed” in this way are referred to as killer cells.

In this respect it is an advantage that the dendritic cells are able to recognize genetically defective cells, transport their freight to the site of action in a targeted manner and inhibit the formation of metastases.

Specification:

Besides about 1,500 scorpion species living all over the world in the moderate zones and the about 35,000 Araneae species, only 70 scolopender species are known. With the exception of approximately 300 cob web spider species, all of the above-mentioned animals are actively poisonous using their poisons for prey catching. Since these animals only have a very small mouth opening, they acquired the capability of pre-digestion outside of the body which is possible due to a highly developed enzyme and poison system. The poison cocktail is either injected directly into the prey animal or the prey is killed mechanically, torn and/or chewed while mixing it with the poison cocktail. The liquefied food with minute solid components can then be aspirated.

About 50 spider species, the same number of scorpion species and about 20 scolopender species can be dangerous to humans due to their poisons. Despite of this, mainly the poisons of the species of medical interest have been investigated only roughly or not at all. The main components of spider poisons are:

digestive enzymes

biogenic amines

organic acids

peptides

peptide toxins.

Among the peptide toxins, the following groups of toxins may be found:

heart toxins

nerve toxins

blood toxins

cell toxins

tissue-destructive toxins.

Initially, the whole poison cocktail of all actively poisonous animals due to a synergistic and/or antagonistic interplay of different substances generally leads to a pre-digestion and thereby a specific alteration of the original animal cells.

In all scolopender, snake, scorpion and spider species used in the present invention the whole poison cocktail contains substances which act in a cytotoxic, necrotic and/or apoptotic manner (digestive action of the poisons). Substances acting as heart toxins, nerve toxins or blood toxins, however, shall be avoided according to the invention. HPLC-MS-MS was used to determine the peptide toxins in the whole poison cocktail. To these were in turn assigned specific modes of action by means of experiments with cells. Now it has been surprisingly found that components of the animal poisons of scolopender centipede species Scolopendra gigantea ssp., Hemiscolopendra spp., of the snake species Bitis arietans (puff adder), Bitis gabonica (Gaboon viper), Bitis nasicornis (rhinoceros viper), and of the smaller Bitis species B. atropos, B. caudalis, B. peringueyi, and of the spitting cobra species Naja melanoleuca, Naja pallida and Naja naja sputatrix, the Parabuthus scorpion species Parabuthus transvallicus, Parabuthus granulosus, Parabuthus villosus, the Loxosceles spider species Loxosceles laeta, Loxosceles spiniceps, Loxosceles bergeri, Loxosceles parami, Loxosceles rufescens, Loxosceles reclusa, Loxosceles deserta, the six eyed sand/crab spider species Sicarius hahni, Sicarius albospinosus, Sicarius oweni, Sicarius testaceus, Sicarius argentinensis, and/or the Pholcus spider species Pholcus phalangioides, Pholcus opilionides, Pholcus spp. (RSA) and Pholcus spp. (Cuba) can be used for the treatment of skin cancer diseases wherein peptide toxins from the poison of scolopenders, Bitis arietans and/or Loxosceles spiniceps are particularly preferred.

Because of the plurality of individual substances and the resulting complex activity within the body, the poison cocktail as a whole cannot be used for pharmaceutical purposes at present. Used as a defense poison by the spiders, one who has been bitten will suffer from the following symptoms:

Scolopenders of the Scolopendra and Hemiscolopendra genera:

Due to their big mouthparts the bite of the animals having a length of up to 30 cm is always noticed. General observations as to the poison effect of giant centipedes (scolopenders) are not available. It is known, however, that the poison is strongly effective in small mammals. Thus, for example the content of one poison gland is sufficient to kill 25 mice with a weight of about 20 g within 7 hours. The poison strongly affects the nervous system leading to the following symptoms: accelerated breathing, sweating, imbalance, vomiting, respiratory paralysis, convulsions, finally death. More recent work with respect to the chemistry, biochemistry, and toxicology of the poisons is not available (Habermehl G. (1987): Gift-Tiere und ihre Waffen, Springer-Verlag, Berlin, 4^{th} and subsequent editions).

Snakes of the Bitis and Naja Genera:

The bite itself is noticed by those bitten due to the impact upon the snake attack. Specifically mentioned is the ability of the spitting cobras to spray the poison via their teeth by means of pressurized air over up to 5 meters well-aimed onto their victims. Immediate pain occurs upon biting due to the poison release. Shortly afterwards the tissue-destructive activity of the poison is initiated. At first starts the local necrotic effect leading to lysis of the skin layers. Subsequently the poison spreads extensively within the tissue surrounding the biting site by means of permeability enhancing enzymes. During this all tissue components with the exception of the skeleton may be destroyed due to necrosis. If the bite occurs into an extremity, an amputation has to be considered depending on the situation. Besides the tissue-destructive toxins there are also found neuro- and cardiotoxins depending on the species which are the primary cause of pain and circulatory instabilities. The main reasons for a lethal outcome of bite accidents are blood poisoning and renal failure.

Scorpions of the Parabuthus Genus:

Due to the neurotoxins contained in the poison cocktail the bite itself already leads to strong pain at the biting site similar to the pain occurring upon burning. After some time the pain is transformed into a tingling sensation which finally becomes a numbness. The general symptoms may occur already after 5 minutes but in some cases not before 24 hours after the bite. The persons bitten are agitated, children may sometimes have convulsions; reflexes are restricted to a minimum or disappear completely. The consciousness remains, however, often accompanied by strong anxiety followed by tear flow and mydriasis while the vision is restricted simultaneously. The pulse is accelerated and irregular. The blood pressure may be either increased or decreased. The body temperature strongly varies while hypothermia is indicative of a worsening of the patient state. Vomiting is a serious indication that also nerve centers are affected. Death occurs primarily by respiratory paralysis, mostly within 20 hours, less frequently within 30 hours (Habermehl G. (1987): Gift-Tiere und ihre Waffen, Springer-Verlag, Berlin, 4^{th} and subsequent editions).

Spiders of the Dysdera Genus:

In contrast to the bite of for example spiders of the Loxosceles and/or Pholcus genus the bite of spiders of the Dysdera genus is in the most cases painfully noticed due to the size and position of their mandibles. After several minutes the biting site becomes red-brown, and blisters occur. During the next hours the skin around the biting site peels up to a size of about 2 cm. An open wound is formed which without treatment often does not heal for weeks. Accompanying symptoms are dyspnoea and cardiac arrhythmias. No cases of death are known.

Spiders of the Loxosceles Genus:

The bite itself is not noticed by the persons bitten in most of the cases. The biting site swells strongly after 2 to 5 hours while the swelling is accompanied by strong pain. The biting site gains a dark red color, and blisters form. Subsequently the color of the tissue turns black and the skin cells necrotize. A hole in the skin remains which may have a diameter of up to 5 cm. Healing may take up to two years where skin transplantation may be necessary in some cases. In severe cases, blood is observed in the urine and eventually renal failure and death occur. In addition there are several cases with hemolytic anemia and hematuria accompanied by sensory disorders. The body temperature may rise up to 41 degrees Centigrade while coma may occur in the severe cases.

Spiders of the Pholcus Genus:

Also in this case, the bite is mostly unnoticed. After about 2 hours the biting site visibly reddens and weeps. In the course of the next hours the skin layer around the biting site starts to detach up to a size of 3 cm. An open wound forms which without treatment does not heal for up to 3 months. Without treatment wound healing is accompanied by strong scar formation. One type of treatment is e.g. skin transplantation. Accompanying symptoms are manifested in circulatory instability. No deaths are known.

However, surprisingly, individual substances of the peptide toxins contained in the animal poison derived from scolopender centipede species Scolopendra gigantea ssp., Hemiscolopendra spp., of the snake species Bitis arietans (puff adder), Bitis gabonica (Gaboon viper), Bitis nasicornis (rhinoceros viper), and of the smaller Bitis species B. atropos, B. caudalis, B. peringueyi, and of the spitting cobra species Naja melanoleuca, Naja pallida and Naja naja sputatrix, the Parabuthus scorpion species Parabuthus transvallicus, Parabuthus granulosus, Parabuthus villosus, the Loxosceles spider species Loxosceles laeta, Loxosceles spiniceps, Loxosceles bergeri, Loxosceles parami, Loxosceles rufescens, Loxosceles reclusa, Loxosceles deserta, the six eyed sand/crab spider species Sicarius hahni, Sicarius albospinosus, Sicarius oweni, Sicarius testaceus, Sicarius argentinensis, and/or the Pholcus spider species Pholcus phalangioides, Pholcus opilionides, Pholcus spp. (RSA) and Pholcus spp. (Cuba) can be used for the treatment of cutaneous carcinoma as well as in parallel or supportive, respectively, in cutaneous carcinoma surgery for the destruction of genetically transformed cells. For example the destruction of melanoma cells and/or of tumor tissue not dissected during the operation may occur according to the present invention by the tracking, unmasking and targeted elimination of the genetically defective cells. In the therapy genetically defective body cells (tumor cells) may be destroyed on the one hand because the surface protein structure and the dendritic cells loaded with peptide toxin employed according to the present invention can recognize and destroy these cells having an altered surface structure. Moreover, the tumor cells secrete specific messengers, so-called tumor markers, to which the dendritic cells loaded with peptide toxin respond after which they destroy the tumor cells. (Zitvogel L. et al. (1999): Novel Immunologic and Therapeutic Attributes of Dendritic cells (DC): Modulation of T and NK cell-Mediated-Antitumor Immune Responses. The European Journal of Cancer, Vol. 35 Supplement 5-S25/Gong J., Avigan D. et al. (1999): Activation of Antitumor Cytotoxic T Lymphocytes by Fusions of Human Dendritic Cells and Breast Carcinoma Cells. The European Journal of Cancer, Vol. 35 Supplement 5-S30).

The mode of action is based on the ability of dendritic cells to track, recognize and destroy genetically defective cells and on the tissue-destructive effect of the peptide toxins as follows:

The peptide toxins having a molecular weight of about 50-350 kDa, in the case of scorpions, spiders and several scolopenders generally of about 100 kDa, have a tissue destructive effect, i.a. also a destructive effect on skin cell types. Because of their high molecular weight and their spatial structure they have only a small spreading tendency within tissues if they are not supported by so-called penetration enzymes.

Dendritic cells are prepared, preferably from spinal cord stem cells because of their uniformity, using a growth factor mixture (GM-CSF/IL-4, preferably in combination with the interleukin receptor sIL-4R) and isolated.

The thus obtained dendritic cells are preferably loaded via lipofection with the individual components having a skin necrotic effect obtained by column chromatography from the poisons of the scolopender centipede species Scolopendra gigantea ssp., Hemiscolopendra spp., of the snake species Bitis arietans (puff adder), Bitis gabonica (Gaboon viper), Bitis nasicornis (rhinoceros viper), and of the smaller Bitis species B. atropos, B. caudalis, B. peringueyi, and of the spitting cobra species Naja melanoleuca, Naja pallida and Naja naja sputatrix, the Parabuthus scorpion species Parabuthus transvallicus, Parabuthus granulosus, Parabuthus villosus, the Loxosceles spider species Loxosceles laeta, Loxosceles spiniceps, Loxosceles bergeri, Loxosceles parami, Loxosceles rufescens, Loxosceles reclusa, Loxosceles deserta, the six eyed sand/crab spider species Sicarius hahni, Sicarius albospinosus, Sicarius oweni, Sicarius testaceus, Sicarius argentinensis, and/or the Pholcus spider species Pholcus phalangioides, Pholcus opilionides, Pholcus spp. (RSA) and Pholcus spp. (Cuba).

The quantity of loading of the dendritic cells may be enhanced by an addition of 1.66% phenyl-Gal-Nac.

Optionally other substances contained in the animal poison raw mixture may promote the effects of the peptide toxins used for loading the dendritic cell.

The mode of action of the dendritic cells loaded with peptide toxins may be carried out by testing these in suitable normal and tumorous human cell lines.

According to the invention the toxic substances or the peptide toxin used for loading of the dendritic cells, respectively, are derived from the poison of scolopenders of the Scolopendridae family, snakes of the Viperidae family, snakes of the Elapidae family, spiders of the Sicariidae family (according to the invention the Sicariidae family includes also the genera Loxosceles and Scytodes besides Sicarius), spiders of the Pholcidae family, scorpions of the Buthidae.

Preferred are the genera Scolopendra and Hemiscolopendra, Bitis and Naja as well as Loxosceles, Pholcus, and Parabuthus. Within the genera Scolopendra and Hemiscolopendra the Scolopendra and Hemiscolopendra giant centipede species Scolopendra morsitans, Scolopendra gigantea and Hemiscolopendra spp., within the Bitis genus the Bitis snake species Bitis arietans, Bitis gabonica and Bitis nasicornis, within the Naja genus the Naja snake species Naja melanoleuca, Naja pallida and Naja naja sputatrix, within the genus Loxosceles the Loxosceles spider species Loxosceles laeta, Loxosceles spiniceps, Loxosceles bergeri, Loxosceles parami, Loxosceles rufescens, Loxosceles reclusa, Loxosceles deserta, within the Sicarius genus the Sicarius spider species Sicarius hahni, Sicarius albospinosus, Sicarius oweni, Sicarius testaceus, Sicarius argentinensis, within the Pholcus genus the Pholcus spider species Pholcus phalangioides, Pholcus spp. (RSA), Pholcus spp. (Cuba) and Pholcus opilionides, within the Parabuthus genus the Parabuthus scorpion species Parabuthus transvallicus, Parabuthus granulosus, Parabuthus villosus may be particularly preferably used. Among the spiders of the Loxosceles genus also the other species may be used according to the invention.

Among the scolopenders of the Scolopendra and Hemiscolopendra genera all species having a body length of more than 10 cm may be used according to the invention.

Among the snakes of the Bitis genus also all dwarf puff adders may be used according to the invention. Among the snakes of the Naja genus also the Asian spitting cobra Naja naja atra may be used according to the invention.

Among the spiders of the Pholcidae family all larger species may be used according to the invention.

Among the scorpions of the Parabuthus genus also all Central African species may be used according to the invention. Besides the scorpions of the Opistophthalmus, Androctonus and Nebo genera may be used according to the invention.

The preparation of the pharmaceutically effective loading according to the present invention may be performed in way that dendritic cells are injected with peptide toxin directly under the microscope via a microcapillary and these cells are then reintroduced into the culture flask. Upon this priming the dendritic cell already loaded with this peptide toxin may communicate its information to the other dendritic cells. Due to this reason, a solution of this peptide toxin used may be added to the medium after about 72 hours. After further 48 hours the dendritic cells have incorporated a certain amount of the peptide toxin depending on the peptide toxin and the dendritic cells.

The preparation may also be performed by inoculating a culture of dendritic cells which may also comprise cutaneous carcinoma cells directly with a peptide toxin destroying the cutaneous carcinoma cells. Because the toxin destroys intact carcinoma cells, specific markers are released upon degradation of the tumor cells signaling to the dendritic cells that the tumor cells destruction can occur more efficiently by means of the peptide toxin while the adaptation of the dendritic cells with the active substances may be enhanced in quantity by addition of phenyl-Gal-Nac.

Due to this fact, another addition in higher concentration of the peptide toxin may be carried out after 24 hours which is then also incorporated by the dendritic cells. It is the aim of the method of preparation to achieve an as high as possible quantitative loading of the dendritic cells with this peptide toxin. At present a determination of the concentration within the cell with measurement techniques is hardly possible or impossible at all. The medium concentrations of the substance to be loaded in the final incubation step generally are in a range of about 0.5 μg/ml to 250 μg/ml depending on the toxin used.

The preparation of the animal poison raw mixture may be preferably performed by obtaining it by methods known per se from Arachnidae and scolopenders and performing a fractionation of the animal poison raw mixture also by fractionation procedures known per se for the separation of proteins to obtain the peptide toxins and/or the other substances having a cell-destructive effect separated from each other in separate fractions if possible. To prepare a pharmaceutically effective loading of dendritic cells individual fractions are preferably used. As the peptide toxins there may be preferably used additionally snake toxins having a cell destructive effect such as e.g. the cobra snake toxin Najatoxin-S, each for the loading of the dendritic cells used in therapy. For the preparation of the pharmaceutically effective fractions of the peptide toxins according to the invention, specific poison components (peptide toxins with necrotic and cytotoxic effect) may be selected e.g. by column chromatographic purification from the poison cocktail which may be obtained by manual milking of the animal species mentioned above. The analysis to distinguish the components contained in the fractions from each other may be carried out using HPLC-MS-MS (e.g. using a device of Perkin-Elmer company). It could be demonstrated that a portion of the high molecular weight substances by the detection of toxic groups of the NH—NHX—NOX and SX types are peptide toxins. This is also confirmed by their mode of action in cell experiments. (Burda R., Schrottenloher E., Weickmann D. (2000): In vitro Versuche zur Anwendung/Verwendung von zellzerstörenden Giftkomponeten der Sechsaugenkrabbenspinnen Sicarius spp. in der biologischen Krebstherapie. Arachnologisches Magazin 2(8):1-7 and Lundblad R. L. (1995): Techniques in Protein Modification, CRC Press, London, Tokyo).

The substances used according to the invention for pharmaceutically effective loading may be obtained in a natural manner. These are poisons produced by giant centipedes of the Scolopendra and Hemiscolopendra genera, from snakes of the Bitis and Naja genera, from cob web spiders of the Loxosceles, Sicarius and Pholcus genera and of scorpions of the Parabuthus genus originally developed for prey catching and pre-digestion of animal protein. This natural mode of action may be obtained by a function-preserving careful preparation of the poison raw substance (e.g. by manual milking). In contrast to conventional arthropod milking methods by means of an electrical procedure (Weickmann D. (1991): Haltung und Giftigkeit von Sicariidae. Arachnologischer Anzeiger 16:12-13; Weickmann D, Burda R. (1994): Electrophoresis of scorpion venoms. Electrophoresis Forum 1994, Abstracts, Techn. Universität München, October 24-26) in which the poison is removed from the animals by an electrical pulse inducing a contraction of the poison glands of the animals (for this purpose, the spiders are preferably hypothermic, scorpions and scolopenders do hardly or not at all release poison during hypothermia), the poison cocktail is obtained according to the present invention by a manual procedure wherein the animals are stimulated to deliver their poison by utilizing their natural defense behavior.

According to one embodiment of the present invention a manual milking method of the spiders is considered. This leads to the preparation of true, pure native poisons while for example in the electrical milking method due to the electron flow restructured substances and molecules, respectively, are obtained from the Arachnidae poisons which may be altered in their mode of action, or substances may be contained in the poisons which the animal normally would not secrete while in the case of giant centipedes no poison is obtained by the conventional electric milking methods. The additional substances secreted by the Arachnidae upon electrical milking may, but must not necessarily have a negative effect on the medical efficiency of the individual compounds contained in the whole poison cocktail. A quality control via electrophoretic procedures of the raw poison may be performed as a standard analysis.

The following Examples shown advantageous embodiments of the invention but, however, are not meant to limit the scope of the invention.

In the Examples and in the Specification, reference is made to the following Figures:

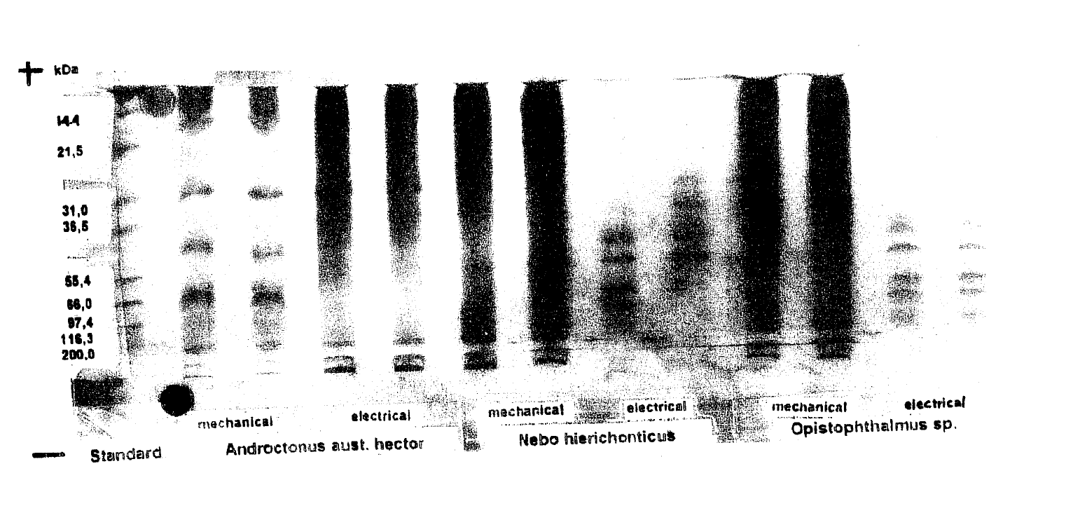

FIG. 1 shows an SDS-PAGE of whole poison cocktails of scorpions obtained by manual and electric milking; lanes from left to right: molecular weight standard; Androctus anst. hector, mechanical milking, electrical milking; Nebo hierchonticus, mechanical milking, electrical milking; Opistophthalmus sp., mechanical milking, electrical milking.

### EXAMPLES

1. Milking:

a) Scolopenders:

For manual milking animals having a body length of more than about 10 cm of the Scolopendra and Hemiscolopendra genera the Scolopendra and Hemiscolopendra giant centipede species Scolopendra morsitans, Scolopendra gigantea and Hemiscolopendra spp. are used. For this purpose the scolopenders are held with the fingers behind the head and fixed with the ball of the thumb in a way that the animal is unable to sting with its last pair of legs transformed into claws. Then the animal is allowed to bite through a plastic wrap tightened around a test tube.

Upon biting the poison is delivered and collects at the test tube. The thus collected poison is placed in an exsiccator which is then stored for at least 12 hours in a deep freezer at a temperature of at least minus 14 degrees Centigrade.

b) Snakes:

Snakes of the Bitis and Naja genera with a body length of about 50 cm are used for milking. For this purpose the snakes are held tightly behind the head and the body is put over the arm. Then the snake is allowed to bite through a plastic wrap tightened around a test tube. Or the snake to be milked is carefully allowed to bite directly into the edge of an beaker or into a vaporization dish. In each case the poison is released upon biting and collects at the bottom of the beaker. The thus collected poison is dried in an exsiccator. The poison dried in this manner may be stored for several years at plus 4 degrees Centigrade.

c) Spiders

For manual milking adult females of the Loxosceles spider species Loxosceles laeta, Loxosceles spiniceps, Loxosceles bergeri, Loxosceles parami, Loxosceles rufescens, Loxosceles reclusa, Loxosceles deserta, within the Sicarius genus the Sicarius spider species Sicarius hahni, Sicarius albospinosus, Sicarius oweni, Sicarius testaceus, within the Pholcus genus the Pholcus spider species Pholcus phalangioides, Pholcus spp. (RSA), Pholcus spp. (Cuba) and Pholcus opilionides were fixed on their backs by the fingers of one hand while with the other hand using a sterile syringe (2 ml Braun Inject of B. Braun company) with adapted sterile needle (20 or 21 by Becton Dickinson) wherein the time of the day is not important at a room temperature of 21 to 27 degrees Centigrade and a humidity of about 60% they were stimulated to secrete the poison at the chelicera by touching with the flat end of the needle. In this respect it is preferred not to exceed a stimulation time of 90 seconds since otherwise the animal is put under unnecessary stress. After the poison drop has appeared at the poison claws it is pulled into the syringe via the needle. A new syringe with new cannula is used for each animal. Afterwards the needle is closed again with its protective needle cap. Immediately afterwards, the closed syringe with the pulled up poison is placed in an exsiccator which is then stored for at least 12 hours in a deep freezer cooled at a temperature of at least minus 14 degrees Centigrade.

d) Scorpions: Animals having a body size of about 3 cm of the scorpion species Parabuthus transvallicus, Parabuthus granulosus and Parabuthus villosus are used for manual milking. For this purpose the scorpions are held at the postabdomen directly behind the poison gland with a pair of tweezers the tips of which are wrapped with foam rubber and the poison sting is preferably contacted with a highly polished aluminium plate (about 4×5 cm) having a thickness of 2 mm. Upon contact, the scorpion secretes a poison drop onto the aluminium plate which is then placed in an exsiccator. This is then stored for at least 12 hours in a deep freezer at a temperature of at least minus 14 degrees Centigrade.

2. Fractionation of the Whole Poison

The deep-frozen whole toxin after removal from the deep freezer is introduced into 1 ml of a solvent e.g. protein solvent from Carl Roth GmbH & Co. KG (solvent for protein column chromatography: 0.25 M Tris/HCl, pH 6.5 to 7.3, 1.92 M glycine in distilled, deionized water (to prevent denaturation no SDS is used in the buffer). This renders poison in solution. The individual poison solutions of the respective species prepared in this way can be collected in a sterile clean teflon vial at room temperature. The sealed teflon vial is then agitated on a vortex mixer for 30 seconds without foam generation thereby obtaining a homogenous solution. After homogenization, the entire solution is introduced via a Perspex funnel (to avoid contamination) into a mounted transparent Perspex column having an inner diameter of 1.5 cm, a wall thickness of 2 mm and a height of 50 cm tapering at the bottom to 1.5 mm which is open and filled with 20 ml of gel (Sigma/Supelco company, AcA 34; matrix: 3% acrylamide/4% agarose; fractionation range (MW): proteins: 20 to 350 kDa; cut off limit: 750 kDa; bead diameter: 60 to 140 micrometers). The thus introduced poison solution passes through the gel and replaces the buffer present in the gel. After the poison solution has completely soaked into the gel further 150 ml of solvent (0.25 M Tris/HCl, pH 6.5 to 7.3, 1.92 M glycine) are loaded onto the column. While passing through the gel this additional solvent replaces the poison solution contained therein. The first 15 ml which elute at the bottom of the column are residual buffer and are discarded. Following this 15 ml, up to 30 fractions depending on the animal species of 4 ml each are collected. The separation into 4 ml fractions was due to the physical and chemical properties of the individual fractions as determined by electrophoresis, preferably SDS-PAGE. As the loading buffers used for the protection of peptide bonds and proteins there are used Roti Load 1+2 (Carl Roth GmbH & Co. KG, Karlsruhe; SDS, glycerol, bromophenol blue, phosphate buffer, Roti Load 1 with mercaptoethanol, Roti Load 2 without mercaptoethanol). The individual fractions are collected separately in sterile clean 5 ml teflon vials with screw caps. Quality control of the individual fractions is carried out by electrophoresis.

The skin cell lysing effect of the fractions was determined by testing the individual fractions from the whole poison cocktail of the respective animal species in an assay using human living cells in highly to excessively dosed amounts (about 200 to 500 μg/ml medium used). Used for loading of dendritic cells were those proteins which destroy both normal skin cells, preferably skin fibroblasts of different biology, and malignant skin cells, preferably 2 lines of malignant melanoma. Not considered were those peptide toxins which besides skin cells also damaged or, in part, even lysed other cells (e.g. breast tissue cells and liver cells).

In the case of Sicarius peptide toxins were obtained in fractions 1-12. These fractions contained peptide toxins in a molecular weight range of about 72 to about 168 kDa. A skin cell lysing, particularly malignant melanoma cell lysing peptide toxin is contained in fraction 10.

For pharmaceutically, therapeutically effective loading of the dendritic cells according to the present invention e.g. individual skin cell lysing fractions of the animal poisons are used. These fractions contained animal poison protein components in a molecular weight range of about 50 to 350 kDa and non-proteinaceous toxins with at least one amido group in a molecular weight range of about 30-55 kDa. An SDS-PAGE of the whole poison cocktail of scorpions is shown in FIG. 1.

To clarify the structure of the individual substances, the respective fractions are investigated by HPLC-MS-MS as well as by DAD-UV spectrometry (DAD or DADI, respectively: Direct Analysis of Daughter Ions). Known substances could not be detected in a higher molecular weight range of more than 10,000. The determinations of the backbone structure indicate that a portion of the substances at least belong to a polypeptide type with toxic components, i.e. polypeptide toxins. However, also toxins having a molecular weight of 30-55 kDa were detected which lack protein structure.

Fractions having the same composition may be collected together. For further processing and storage the individual fractions are lyophilized, using for example the following parameters:

The fraction to be lyophilized is cooled to minus 22 degrees Centigrade in an open teflon vial loosely covered with perforated aluminium foil. To ensure that the sample is completely frozen a cooling period of 11 hours (at least 20 hours in the case of scolopenders) is kept. Then, a vacuum of 0.200 mbar is produced. After the vacuum is reached the fraction is warmed up to plus 4 degrees Centigrade and kept at this temperature for at least 24 hours while maintaining the vacuum. After completion of the lyophilization procedure, the teflon vial containing the lyophilized fraction is screwed up airtight. The storage stability at room temperature is about 3 months (4 weeks in the case of scolopender poisons), about 1 year at plus 7 degrees Centigrade (at this temperature scolopender poisons are only stable for about 5 months), and about 5 years at minus 14 degrees Centigrade (for a prolonged storage scolopender poisons should be stored at minus 80 degrees Centigrade).

3. Loading of Dendritic Cells with Peptide Toxins

3-5 ml of a culture of dendritic cells (2.5 to 15 millions of dendritic cells/5 ml) prepared according to the method described above in the present invention were contacted with 0.5 to 2.5 ml of a cutaneaous carcinoma cell solution (supernatant of a freshly tapped pure human cutaneous carcinoma cell culture flask containing about 250,000 cutaneous carcinoma cells/ml cell medium. Medium: 500 ml DMEM Ham's F-12, 10 ml penicillin/streptomycin, 5 ml glutamine and 50 ml FBS. Afterwards 1 to 2 ml of a solution containing about 150 μg/ml peptide toxin of fraction 3 derived from Loxosceles spiniceps were added. Incubation was carried out for 24 hours at room temperature. Then, using a previously autoclaved Pasteur pipette, about 4 ml of medium supernatant (if possible without dendritic cells) were removed under a sterile workbench and discarded. Subsequently another addition of 1 to 2 ml of the peptide toxin Lox.tox. 3 (same concentration as in the first addition) was performed resulting in a final concentration of peptide toxin of about 100 μg/ml. The dendritic cells loaded in this way are further incubated at 37° C. in an incubator until they are used.

4. Effect of the Loaded Dendritic Cells on Malignant Melanoma Cells

The dendritic cells loaded with the above-mentioned toxins are collected/enriched to generate an as “saturated” solution of dendritic cells loaded with toxins as possible. In a mixed tissue cell experiment, i.e. a culture of normal tissue and cutaneous tumor cells (cell culture: malig. melanoma: cells truly identified as such obtained from the patient by arrangement, also studied in long-term culture and subculture since 1995), the effects of various toxins used to load the dendritic cells were tested. On average the tumor cell areas (malig. melanoma cells) had a size of 4 mm and were about 1-2 mm thick. Loaded dendritic cells were added directly to the medium (500 ml DMEM Ham's F-12, 10 ml penicillin/streptomycin; 5 ml glutamine and 50 ml FBS), a procedure which although all tumor cells areas were attacked took the longest time and had to be repeated after 3 weeks. Injection into dendritic cells may be carried out very selectively but many dendritic cells die upon this procedure. It was found to be most effective to inject the cells loaded with toxins directly at the tumor edge so that according to their natural behavior the dendritic cells can first destroy the biologically active tumor edge. The lysed tumor cells are digested (partially by macrophages). For all animal toxins examined the lysed cell areas were again overgrown with normal cells. In this Example the effect of the following toxins was examined:

| | | |
|---|---|---|
| | Fractions of the | |
| | molecular weight | |
| | of the whole | |
| Toxins from: | poison cocktail | in kDa |

| | | |
|---|---|---|
| Scolopendra morsitans C.S.A. | 2 | 130 |
| Hemiscolopendra sp. ex Peru | 2 | 140 |
| Bitis arietans | 4 | 125 |
| Bitis gabonica | 4 | 210 |
| Loxosceles laeta | 3 | 160 |
| Loxosceles spiniceps | 3 | 175 |
| Pholcus phalangioides | X | 115 |
| Parabuthus transvallicus | 17 | 305 |

X: may bands closely together on the gel which thus cannot be unambiguously assigned to fraction 5 or 6.

The best and quickest tumor destruction already after a single administration of dendritic cells at the tumor edge was obtained with toxins from scolopenders, Bitis arietans, Loxosceles spiniceps. In these cases complete tumor cell destruction after only 6 h after a single addition was achieved while the lysed cell areas were overgrown with normal cells.

If not all tumor cells are destroyed a new addition of loaded dendritic cells is performed every two days.

5. Effect of the Loaded Dendritic Cells on Liver Tumor Cells

Experiments were carried out with respect to the effect of dendritic cells loaded with liver cell destructive toxins from:

Bitis atropos molecular weight of 13 kDa and/or

Naja naja sputatrix molecular weight of 18 kDa and/or

Hemiscolopendra spp. molecular weight of 102 kDa and/or

Loxosceles reclusa molecular weight of 113 kDa and/or

Sicarius hahni molecular weight of 192 kDa and/or

Pholcus phalangioides molecular weight of 90 kDa and/or

Dysdera crocata molecular weight of 102 kDa and/or

on liver tumor cells.

The dendritic cells transport the toxic substances mentioned above into the proximity of the malignant liver tumor cells and always lead to the destruction of the malignant tumorous liver cells. The experimental setting and protocol were as described under 4. using a mixed culture of liver cells and liver tumor cells (both liver primary tumor cells and liver metastases cells) instead of skin and melanoma cells. The addition of the dendritic cells loaded with liver cell destructive toxins to the mixed tissue cultures was also performed as described under 4.

1. Dendritic cells loaded with toxic substances characterized in that said toxic substances (toxins) are selected from the poison of scolopenders of the genera Scolopendra and Hemiscolopendra, snakes of the genera Bitis and Naja, spiders of the genera Loxosceles, Sicarius, Pholcus and Dysdera as well as scorpions of the genus Parabuthus and a combination of one or more of these toxins.

2. Dendritic cells loaded with toxic substances characterized in that said substances are selected from toxins from poisons of the scolopender species Scolopendra morsitans, S. gigantea and Hemiscolopendra sp., the snake species Bitis arietans (puff adder), Bitis gabonica (Gaboon viper), Bitis nasicornis (rhinoceros viper), B. atropos, B. caudalis, B. peringueyi, the spitting cobra species Naja melanoleuca, Naja pallida and Naja naja sputatrix, the araneomorphic violin spider species Loxosceles laeta, L. spiniceps, L. bergeri, L. parami, L. rufescens, L. reclusa, L. deserta, the six eyed sand/crab spider species Sicarius hahni, S. albospinosus, S. testaceus, S. argentinensis, the vibrating spider species Pholcus phalangioides, Pholcus opilionides, Pholcus spp. (RSA) and Pholcus spp. (Cuba), the fat tail scorpion species Parabuthus transvallicus, P. granulosus, P. villosus, and a combination of one or more of these toxins.

3. Dendritic cells according to claim 1
characterized in that the dendritic cells are of human origin.

4. Dendritic cells according to claim 1 or 2
characterized in that the toxins have been obtained by fractionation of the poison cocktail of the animal species mentioned.

5. Dendritic cells according to one or more of-the preceding claims
characterized in that the toxins obtained by fractionation of the poison cocktail are used together with other substances present in the individual fractions without carrying out further purification.

6. Dendritic cells according to one or more of the preceding claims
characterized in that the toxins are used in pure form.

7. Dendritic cells according to one or more of the preceding claims
characterized in that the toxins are present in recombinant form.

8. Dendritic cells according to one or more of the preceding claims
characterized in that the toxins are peptide toxins.

9. Dendritic cells according to one or more of the preceding claims
characterized in that the peptide toxins have a molecular weight of about 50-350 kDa.

10. Dendritic cells according to one or more of the preceding claims
characterized in that the toxins have necrotic, cytotoxic and/or apoptotic properties.

11. Dendritic cells according to one or more of the preceding claims
characterized in that the dendritic cells have been loaded with the toxins by lipofection and/or laser microinjection.

12. Dendritic cells according to one or more of the preceding claims
characterized in that the toxins have an amido group but no protein structure and a molecular weight of 30-55 kDa.

13. Dendritic cells according to one or more of the preceding claims
characterized in that the dendritic cells are loaded with the toxins in a therapeutically effective amount wherein for the loading the dendritic cells have been contacted with a medium wherein the toxin concentration is in the range of 0.5-250 μg/ml, preferably 80-225 μg/ml, even more preferred about 170 μg/ml.

14. Dendritic cells according to one or more of the preceding claims
characterized in that peptide toxins prepared synthetically or by recombination are used.

15. Dendritic cells according to one or more of the preceding claims
characterized in that derivatives of the peptide toxins are used wherein one or more amino acids are added, deleted and/or substituted wherein the toxic properties of the toxin are substantially retained.

16. Dendritic cells according to one or more of the preceding claims
characterized in that they are further loaded with interferons, cytokins, cytokin receptors, anaphylatoxins, glycosides and/or antibodies.

17. A method for the preparation of dendritic cells according to one or more of the preceding claims characterized in that the dendritic cells have been contacted with one or more toxins as defined in claims 1-16 to associate the cells with the toxins.

18. A method according to claim 17
characterized in that for the loading the dendritic cells are incubated with the toxic substances.

19. A method for the preparation of dendritic cells according to one or more of the preceding claims characterized in that the toxic substances are incorporated into the dendritic cells or associate with the dendritic cells.

20. A method for the preparation of dendritic cells according to one or more of the preceding claims characterized in that loading is performed by lipofection and/or laser microinjection of the toxic substances.

21. The use of the dendritic cells loaded with toxic substances according to one or more of the preceding claims for the treatment of tumor diseases, particularly tumors of the skin and mucosa.
